(19) United States
(12) Patent Application Publication
(10) Pub. No.: US 2013/0059812 A1
(43) Pub. Date: Mar. 7, 2013

(54) COMPOSITION FOR TREATING CHRONIC HEPATITIS B, CONTAINING CLEVUDINE AND ADEFOVIR DIPIVOXIL

(71) Applicants: Sung-Koo Lee, Seoul (KR); Young-Choon Lee, Seoul (KR)

(72) Inventors: Sung-Koo Lee, Seoul (KR); Young-Choon Lee, Seoul (KR)

(73) Assignee: BUKWANG PHARM CO LTD, SEOUL (KR)

(21) Appl. No.: 13/697,667

(22) PCT Filed: Apr. 18, 2011
(86) PCT No.: PCT/KR11/02747

§ 371 Date: Nov. 13, 2012

(30) Foreign Application Priority Data
May 18, 2010 (KR) 10-2010-0046543

(51) Int. Cl. A61K 31/7072; A61P 31/20

(52) U.S. Cl. 514/50

(57) ABSTRACT

The present invention relates to a composition for treating chronic hepatitis B, containing clevudine and adefovir dipivoxil. The combined formulation of the present invention maximizes the effect for treating diseases caused by infection of hepatitis B virus and shows a mutual inhibitory activity against a resistant virus compared with a single-component formulation.

### TECHNICAL FIELD

The present invention relates to a composition for treating chronic hepatitis B, containing clevudine and adefovir dipivoxil. The combined formulation of the present invention maximizes the effect of treating diseases caused by infection of hepatitis B virus and shows a mutual inhibitory activity against a resistant virus compared with a single-component formulation.

### BACKGROUND ART

Hepatitis is an inflammation of the liver, leading to disruption of hepatocytes, the most frequent cause of which is considered to be viral infection. Hepatitis B, the most frequent viral hepatitis, is scientifically defined as the condition that the viral surface antigen (HBsAg) retains for more than six months. Hepatitis B virus (HBV) replicates after the infection into cells and then secretes surface antigen, envelope antigen (HbeAg) and virion containing its DNA. Accordingly, the antigens are used as indicators in the diagnosis of hepatitis.

In acute hepatitis, clinical symptoms such as hypohepatia are cured within three or four months after the occurrence of hepatitis. On the other hand, chronic hepatitis means the condition that hepatitis lasts six months or longer in the diagnosis of liver function and biopsy. It is reported that about 25% of chronic HBV carriers die of primary HCC or hepatocirrhosis.

According to a report from the World Health Organization, there are about 400 million chronic hepatitis B carriers world wide, about 1 million or more of whom die per year. In 20% of patients, chronic hepatitis B is transformed to liver cancer or hepatocirrhosis within 10˜20 years after infection, and 70% of patients with liver cancer have correlations with infection with hepatitis B virus. Accordingly, it is highly necessary to develop effective treatment for suppressing the replication of hepatitis B virus.

The medicine developed for treating chronic hepatitis B until now cannot eliminate HBV completely from the body. Furthermore, such medicine has a problem that long-term use always results in the appearance of mutant HBV resistant to the medicine.

One of nucleosides, 1-(2′-deoxy-2′-fluoro-β-L-arabinofuranosyl)thymine (hereinafter, “clevudine”) is commercially available by the tradename of “Levovir.” Clevudine is known to have an advantage of reducing cccDNA in hepatocytes of patients with chronic hepatitis B, which maintains the antiviral activity for a long time after the termination of administration thereof.

An antiviral agent, adefovir dipivoxil, also referred to as 9-[2-[bis(pivaloyloxy)-methoxy]phosphinylmethoxyethyl]adenine, is a reverse transcriptase inhibitor and has superior antiviral activity against HIV and HBV in vivo. As for the antiviral activity of adefovir dipivoxil, refer to Barditch-Crovo P. et al., J. Infect. Dis., 176(2):406 (1997) and Starrett et al., J. Med. Chem., 37:1857-1864 (1994).

Adefovir dipivoxil is known to have two forms of amorphism and crystal form in the natural state, and has a problem of decreased stability due to the formation of hydrolyzed products by moisture. In order to solve the problem of stability, a PCT application (WO 0035460A) provides more stable pharmaceutical compositions comprising anhydrous crystalline adefovir dipivoxil, crystalline adefovir dipivoxil dehydrate, and alkaline excipient.

For effective long-term therapy to treat chronic hepatitis B, combined administration of antiviral agents is required. The most serious problem of antiviral agents against HBV is an appearance of drug-resistant virus a certain period after administration of the antiviral agents. In general, the most common drug-resistant virus is a lamivudine-resistant virus which has rtL180M mutation in HBV polymerase B domain and rtM204I/V mutation in C domain. If the lamivudine-resistant virus starts to appear in a patient who has been administered lamivudine, the blood virus concentration increases with a sharp rise in ALT/AST, which makes the patient's condition worse. In this case, it is necessary to administer other antiviral agents to which the resistant virus is susceptible. Among the commercially available antiviral agents for treating chronic hepatitis B, adefovir dipivoxil is effective against the lamivudine-resistant virus. However, adefovir dipivoxil does not have sufficient antiviral activity, and long-term administration thereof results in appearance of adefovir dipivoxil-resistant virus, which has rtA181V/T and rtN236T mutations in HBV polymerase domain. Accordingly, in order to suppress the expression of resistant virus to a specific antiviral agent and to effectively treat chronic hepatitis B, it is necessary to administer combined antiviral agents showing a mutual inhibitory activity against drug-resistant virus, rather than a single agent.

U.S. Pat. No. 6,528,515 discloses a method for treating hepatitis B virus infection by administering a combination of agents having known anti-hepatitis B virus activity, and mentions a combination of adefovir dipivoxil and clevudine as an example. The patent states that the potency of the mixture was lowest at the highest relative concentration of clevudine (1:1 molar ratio), and the most favorable overall interactions were observed at the 3:1 molar ratio of the two compounds. However, it only exemplifies the combined use of clevudine and adefovir dipivoxil but presents no experimental data supporting the effect obtained from the combined use. Moreover, it never mentions a drug-resistant virus having mutations.

Considering the above, the present inventors have conducted research on a combination therapy of an antiviral agent against HBV to maximize the effect on the hepatitis B virus infection and developed a novel composition especially having an inhibitory effect against drug-resistant mutant virus.

### CONTENTS OF THE INVENTION

### Problems to be Solved

The object of the present invention is to provide a composition which maximizes the effect of treating diseases caused by the infection of hepatitis B virus and shows a mutual inhibitory activity against a resistant virus.

### Technical Means

Therefore, the present invention provides a composition for treating chronic hepatitis B virus infection, comprising clevudine and adefovir dipivoxil.

According to one aspect of the present invention, a composition comprising clevudine and adefovir dipivoxil at a weight ratio of 3:1˜1:1 is provided. The potency test showed that the composition of the present invention has a synergistic effect on treating hepatitis B virus infection, that is superior to those of a single compound, and that the effect increases with the increase of the concentration of clevudine in the composition. Especially, the composition comprising clevudine and adefovir dipivoxil has a synergistic effect against mutant virus having a resistance to lamivudine and adefovir, which supports the superior inhibitory activity of the composition of the present invention against mutant virus.

The dosage of the composition for treating chronic hepatitis B virus infection according to the present invention can be adjusted depending on the ages or conditions of patients, formulations to be administered or kinds of drugs. Preferably, the composition comprises 5˜100 mg of clevudine and 5˜30 mg of adepovir dipivoxil; more preferably, the composition comprises 10˜30 mg of clevudine and 5˜10 mg of adepovir dipivoxil.

The composition of the present invention can be prepared by using pharmaceutically acceptable excipients and a conventional manufacturing method to achieve high bioavailability.

According to the present invention, the pharmaceutically acceptable carriers are organic or inorganic carriers for formulating a solid medicament, which include, for example, excipients, lubricants, binders and disintegrating agents.

Preferable excipients include, for example, lactose, sugar, D-mannitol, D-sorbitol, corn starch, dextrin, crystalline cellulose, low-substituted hydroxypropyl cellulose, sodium carboxymethyl cellulose (CMC-Na), arabic gum, amylopectin, light anhydrous silicic acid, synthetic aluminum silicate, aluminum-magnesium silicate, etc.

Preferable lubricants include, for example, magnesium stearate, calcium stearate, talc, silica gel, etc. Preferable binders include, for example, povidone, pregelatinized starch, corn starch, sucrose, glutin, arabic gum, methyl cellulose, carboxymethyl cellulose, sodium carboxymethyl cellulose, crystalline cellulose, sugar, hydroxypropyl cellulose, hydroxypropylmethyl cellulose (HPMC), etc.

Preferable disintegrating agents include, for example, sodium starch glyconate, calcium carboxymethyl cellulose, sodium carboxymethyl cellulose, light anhydrous silicic acid, low-substituted hydroxypropyl cellulose, etc.

### Effect of the Invention

The combined formulation of the present invention comprising clevudine and adefovir dipivoxil maximizes the effect for treating hepatitis B virus infection and shows a mutual inhibitory activity against a resistant virus compared with a single-component formulation.

### BRIEF DESCRIPTION OF DRAWINGS

FIG. 1 is the result of southern hybridization which shows the effects of clevudine, adefovir dipivoxil and a combined formulation of clevudine and adefovir dipivoxil against wild type HBV.

FIG. 2 is the result of a phosphor imager which shows the effects of clevudine, adefovir dipivoxil and a combined formulation of clevudine and adefovir dipivoxil against wild type HBV.

FIG. 3 is the result of southern hybridization which shows the effects of clevudine, adefovir dipivoxil and a combined formulation of clevudine and adefovir dipivoxil against patient-derived rtM204I mutant virus.

FIG. 4 is the result of a phosphor imager which shows the effects of clevudine, adefovir dipivoxil and a combined formulation of clevudine and adefovir dipivoxil against patient-derived rtM204I mutant virus.

### CONCRETE EXPLANATION TO CARRY OUT THE INVENTION

The present invention is explained in more detail by the following Examples. However, these Examples seek to illustrate the present invention only, and the scope of the present invention is not limited thereto.

### Example 1

### Preparation of Tablets Containing Clevudine and Adefovir Dipivoxil

[Ingredients]

| | |
|---|---|
| Clevudine | 30 mg |
| Adefovir dipivoxil | 10 mg |
| Sodium starch glyconate | 10 mg |
| Povidone | 1 mg |
| D-mannitol | 142 mg |
| Talc | 5 mg |
| Magnesium stearate | 2 mg |

Adefovir dipivoxil, clevudine, sodium starch glyconate, povidone and D-mannitol were mixed after being sieved by an 18-mesh sieve, and the mixture was granulated using water. The granules were dried until the moisture content thereof was less than 2% (KF method). The dried granules were sieved by a 25-mesh sieve and then mixed with talc and magnesium stearate. The mixture was pressed by a flat circular punch pin with a diameter of 8 mm to produce tablets of 200 mg.

### Example 2

### Preparation of Tablets Containing Clevudine and Adefovir Dipivoxil

[Ingredients]

| | |
|---|---|
| Clevudine | 20 mg |
| Adefovir dipivoxil | 10 mg |
| Sodium starch glyconate | 10 mg |
| Povidone | 1 mg |
| D-mannitol | 152 mg |
| Talc | 5 mg |
| Magnesium stearate | 2 mg |

Adefovir dipivoxil, clevudine, sodium starch glyconate, povidone and D-mannitol were mixed after being sieved by an 18-mesh sieve, and the mixture was granulated using water. The granules were dried until the moisture content thereof was less than 2% (KF method). The dried granules were sieved by a 25-mesh sieve and then mixed with talc and magnesium stearate. The mixture was pressed by a flat circular punch pin with a diameter of 8 mm to produce tablets of 200 mg.

### Example 3

### Preparation of Tablets Containing Clevudine and Adefovir Dipivoxil

[Ingredients]

| | |
|---|---|
| Clevudine | 10 mg |
| Adefovir dipivoxil | 10 mg |
| Sodium starch glyconate | 10 mg |
| Povidone | 1 mg |
| D-mannitol | 162 mg |
| Talc | 5 mg |
| Magnesium stearate | 2 mg |

Adefovir dipivoxil, clevudine, sodium starch glyconate, povidone and D-mannitol were mixed after being sieved by an 18-mesh sieve, and the mixture was granulated using water. The granules were dried until the moisture content thereof was less than 2% (KF method). The dried granules were sieved by a 25-mesh sieve and then mixed with talc and magnesium stearate. The mixture was pressed by a flat circular punch pin with a diameter of 8 mm to produce tablets of 200 mg.

### Experimental Example 1

### Antiviral Efficacy Test of a Combined Preparation of Clevudine and Adefovir Dipivoxil Against Wild-Type HBV

In order to maximize the effect of treating chronic hepatitis B, administration of a combined preparation of antiviral agents is required. The following test was carried out to confirm the antiviral effect of the combined preparation of clevudine and adefovir dipivoxil.

(1) Viral DNA Transfection

About 20˜24 hours before the transfection, Huh-7 cell lines were maintained on 6-well plates to be 4×10⁵ cells/well with 70˜80% confluence and then cultured. 2 hours before the transfection, the culture medium was removed from the stabilized cells and replaced with a serum-free medium containing clevudine, adefovir dipivoxil or a combined preparation of clevudine and adefovir dipivoxil in a predetermined dilution to be 2 mL/well. Using lipofectamine (Invitrogen), the cells were transfected with wild-type HBV DNA to be 2 μg per well. The concentration of antiviral agents was 0.9 μM of clevudine, 6.3 μM of adefovir dipivoxil, 0.9 μM+6.3 μM of clevudine+adefovir dipivoxil or 1.8 μM+6.3 μM of clevudine+adefovir dipivoxil. About 5 hours after the transfection, the culture medium was replaced with that treated with the antiviral agents, and then the medium was replaced every 24 hours.

(2) Collection of Cells and Separation of HBV DNA

On the 4^{th} day of the transfection, the cells were washed with cold PBS and then collected. The collected cells were lysed with cold Hepes lysis buffer (1% NP-40) and then centrifuged. The supernatant was treated with nuclease to remove DNA of the transfected cells. Core protein of capsid form was precipitated with 26% PEG solution. The capsid protein was removed by 0.5% SDS solution and Proteinase K, and then HBV DNA in the capsid was separated.

(3) Southern Hybridization

Electrophoresis was carried out with the separated HBV DNA on 1% agarose gel, which was then transferred to a positively charged nylon membrane. Gel-purified ³²P-labeled whole-length HBV fragment was hybridized with the transferred membrane, which was washed with SSC buffer and then exposed to X-ray film. An antiviral effect of the combined formulation of clevudine and adefovir dipivoxil was determined using phosphor imager compared with single treatment of clevudine or adefovir dipivoxil.

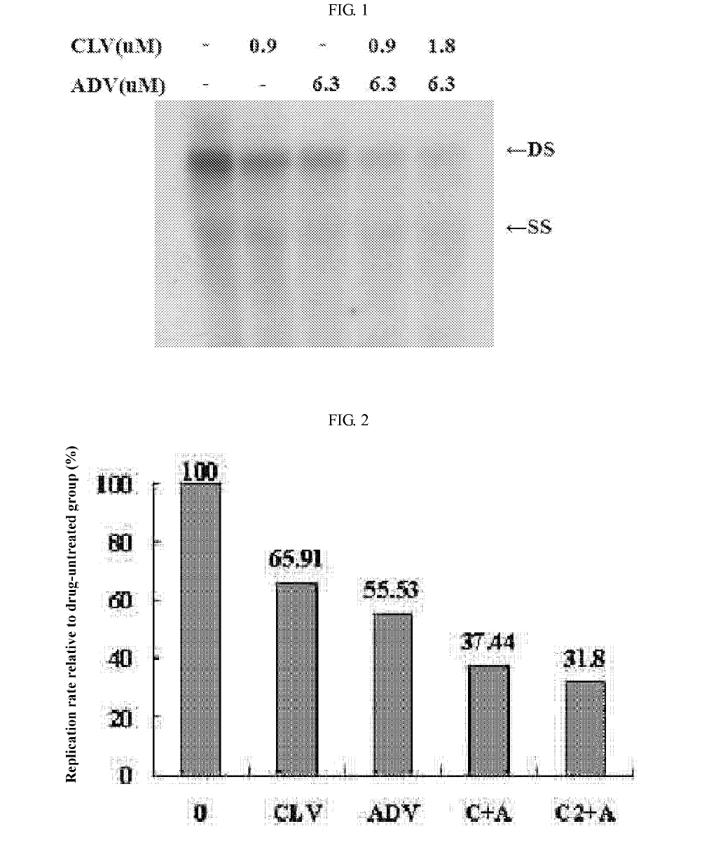

FIG. 1 is the result of southern hybridization which shows the effects of clevudine, adefovir dipivoxil and the combined formulation of clevudine and adefovir dipivoxil against wild type HBV, and FIG. 2 is the result of phosphor imager.

As shown in FIGS. 1 and 2, the combined formulation of clevudine and adefovir dipivoxil according to the present invention has effective antiviral activity against wild-type HBV compared with single treatment of clevudine or adefovir dipivoxil. Specifically, when the rate of wild-type HBV replication is 100% in the untreated control group, the rates of wild-type HBV replication were 65.91% and 55.53% in the clevudine-treated group and adefovir dipivoxil-treated group, respectively, while the rate of wild-type HBV replication was 37.44% in the group treated with the combined formulation of clevudine and adefovir dipivoxil. That is, the inhibition rate of wild-type HBV replication was 62.56% in the group treated with the combined formulation of clevudine and adefovir dipivoxil, which is about a two-fold antiviral effect compared with the inhibition rate of 34.09% in the clevudine-treated group. Accordingly, the combined formulation of clevudine and adefovir dipivoxil shows antiviral activity against wild-type HBV superior to that with a single formulation of clevudine or adefovir dipivoxil.

### Experimental Example 2

### Antiviral Efficacy Test of a Combined Preparation of Clevudine and Adefovir Dipivoxil Against Mutant HBV

As mentioned above, the most serious problem of antiviral agents against HBV is an appearance of drug-resistant virus a certain period after administration of the antiviral agents. In general, the most popular drug-resistant virus is a lamivudine-resistant virus which has rtL180M mutation in HBV polymerase B domain and rtM204I and rtM204V mutation in C domain. As adefovir dipivoxil-resistant virus, rtA181V, rtA181T and rtN236T mutation in HBV polymerase domain are known. According to the present invention, a combined formulation of adefovir dipivoxil effective against the most common lamivudine-resistant virus and clevudine effective on adefovir dipivoxil-resistant virus is provided. The following test was carried out for confirming the antiviral effect of the combined preparation of clevudine and adefovir dipivoxil against drug-resistant virus.

(1) Cloning of Mutant Virus

For cloning of lamivudine-resistant HBV and adefovir dipivoxil-resistant HBV having a mutation in HBV polymerase domain, HBV DNA was separated from the serum of a patient (QIAmp MinElute Virus Spin Kit, QIAGEN). Reverse transcriptase domain of HBV was amplified by PCR and then sub-cloned to a wild-type whole-gene expression vector. DNA was separated using a kit (Axygen).

(2) Viral DNA Transfection

About 20˜24 hours before the transfection, Huh-7 cell lines were maintained on 6-well plates to be 4×10⁵ cells/well with 70˜80% confluence and then cultured. 2 hours before the transfection, the culture medium was removed from the stabilized cells and replaced with a serum-free medium containing clevudine, adefovir dipivoxil or a combined formulation of clevudine and adefovir dipivoxil in a predetermined dilution to be 2 mL/well. Using lipofectamine (Invitrogen), the cells were transfected with HBV mutant DNA to be 2 μg per well. The concentration of antiviral agents was 0.9 μM of clevudine, 6.3 μM of adefovir dipivoxil, 0.9 μM+6.3 μM of clevudine+adefovir dipivoxil or 1.8 μM+6.3 μM of clevudine+adefovir dipivoxil. About 5 hours after the transfection, the culture medium was replaced with that treated with the antiviral agents, and then the medium was replaced every 24 hours.

(3) Collection of Cells and Separation of HBV DNA

On the 4^{th} day of the transfection, the cells were washed with cold PBS and then collected. The collected cells were lysed with cold Hepes lysis buffer (1% NP-40) and then centrifuged. The supernatant was treated with nuclease to remove DNA of the transfected cells. Core protein of capsid form was precipitated with 26% PEG solution. The capsid protein was removed by 0.5% SDS solution and Proteinase K and then HBV DNA in the capsid was separated.

(4) Southern Hybridization

Electrophoresis was carried out with the separated HBV DNA on 1% agarose gel, which was then transferred to a positively charged nylon membrane. Gel-purified ³²P-labeled whole-length HBV fragment was hybridized with the transferred membrane, which was washed with SSC buffer and then exposed to X-ray film. An antiviral effect of the combined formulation of clevudine and adefovir dipivoxil was determined using phosphor imager compared with the single treatment of clevudine or adefovir dipivoxil.

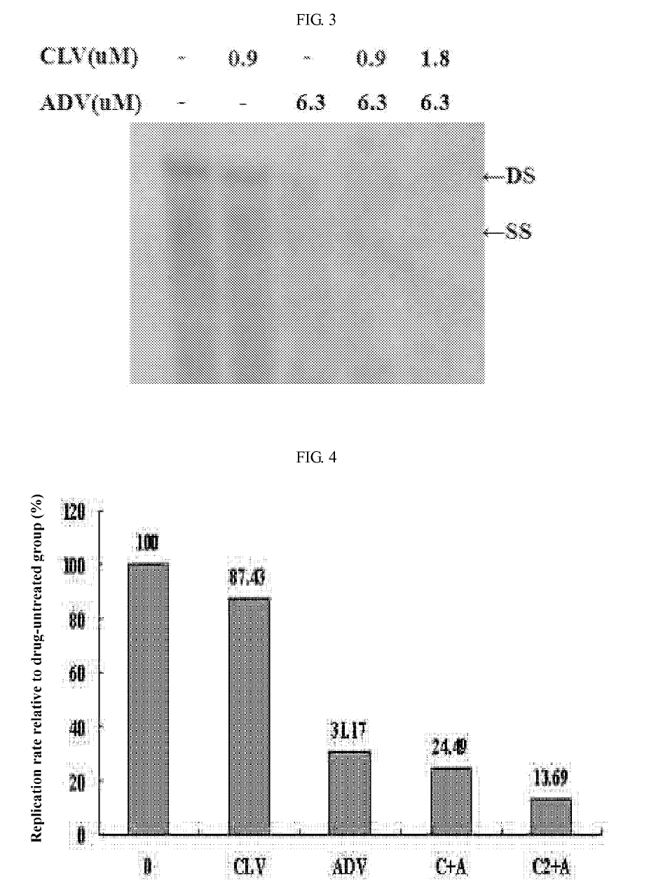

FIG. 3 is the result of southern hybridization which shows the effects of clevudine, adefovir dipivoxil and the combined formulation of clevudine and adefovir dipivoxil against patient-derived rtM204I mutant virus, and FIG. 4 is the result of phosphor imager.

As shown in FIGS. 3 and 4, the combined formulation of clevudine and adefovir dipivoxil according to the present invention has a more effective antiviral activity against rtM204I mutant virus than single treatment of clevudine or adefovir dipivoxil. Specifically, when the rate of patient-derived rtM204I mutant virus replication is 100% in the untreated control group, the rates of mutant virus replication were 87.43% and 31.17% in the clevudine-treated group and adefovir dipivoxil-treated group, respectively, while the rate of mutant virus replication was 24.49% in the group treated with the combined formulation of clevudine and adefovir dipivoxil. In addition, the rate of mutant virus replication was 13.69% in the group treated with the combined formulation with the amount of clevudine doubled. That is, the inhibition rate of mutant virus replication was 75.51% and 86.31% in the group treated with the combined formulation of clevudine and adefovir dipivoxil, which is even more effective than the inhibition rate of 68.83% in the adefovir dipivoxil-treated group, known as being effective on mutant virus.

### Experimental Example 3

### Antiviral Efficacy Test of Combined Preparation of Clevudine and Adefovir Dipivoxil in the Treatment of Chronic Hepatitis B Patients

Combination therapy of two antiviral agents is a new clinically applicable strategy. Nonetheless, the combined formulation of clevudine and adefovir dipivoxil has not yet been studied. The following test was carried out for confirming the antiviral effect of the combined preparation of clevudine and adefovir dipivoxil according to the present invention in the treatment of chronic hepatitis B patients.

Antiviral Efficacy Test in the Treatment of Chronic Hepatitis B Patients

A total of 40 patients were divided into two groups, to one of which 30 mg of clevudine was administered daily for 24 weeks (group treated with clevudine only; n=20). To another group, a combined formulation of 30 mg of clevudine and 10 mg of adefovir dipivoxil was administered daily for 12 weeks, then administration of adefovir dipivoxil was stopped and only 30 mg of clevudine was administered daily for the remaining period (group treated with combined formulation of clevudine+adefovir dipivoxil; n=20).

As a result, 12 weeks after the administration, the number of HBV DNA copy was −2.67 log 10 copies/mL in the group treated with clevudine only, and −4.11 log 10 copies/mL in the group treated with the combined formulation of clevudine+adefovir dipivoxil, which is significantly different (p=0.001). 24 weeks after the administration, the number of HBV DNA copy was −4.15 log 10 copies/mL in the group treated with clevudine only, and −4.97 log 10 copies/mL in the group treated with the combined formulation of clevudine+adefovir dipivoxil, which is significantly different (p=0.036).

The example showed that in the initial therapy of chronic hepatitis B patients, the administration of the combined formulation of clevudine and adefovir dipivoxil can effectively suppress the HBV replication compared with that of a single formulation of clevudine.

### INDUSTRIAL APPLICABILITY

The combined formulation of the present invention comprising clevudine and adefovir dipivoxil maximizes the effect of treating hepatitis B virus infection and shows a mutual inhibitory activity against a resistant virus compared with a single-component formulation. Accordingly, the combined formulation of the present invention is especially useful for the treatment of chronic hepatitis B requiring long-term therapy.

1-9. (canceled)

10. A method of treating a chronic hepatitis B infection in a patient in need thereof comprising administering to said patient clevudine and adefovir dipivoxil.

11. The method of claim 10, comprising administering from 5 to 100 mg of clevudine and from 5 to 30 mg of adefovir dipivoxil.

12. The method of claim 10 comprising administering from 10 to 30 mg of clevudine and from 5 to 10 mg of adefovir dipivoxil.

13. The method of claim 10, wherein the chronic hepatitis B virus is a drug-resistant virus.

14. The method of claim 10 wherein the chronic hepatitis B virus is a lamivudine-resistant virus.

15. The method of claim 10 wherein the chronic hepatitis B virus is a lamivudine-resistant virus having an rtL180M, rtM204I or rtM204V mutation.

16. The method of claim 10 wherein the chronic hepatitis B virus is an adefovir dipivoxil-resistant virus.

17. The method of claim 10 wherein the chronic hepatitis B virus is an adefovir dipivoxil-resistant virus having an rtA180V, rtA181T or rtN236T mutation.

18. A method of treating a lamivudine resistant chronic hepatitis B infection having an rtL180M, rtM204I or rtM204V mutation in a patient in need thereof comprising administering to said patient clevudine and adefovir dipivoxil.

19. The method of claim 18, comprising administering from 5 to 100 mg of clevudine and from 5 to 30 mg of adefovir dipivoxil.

20. The method of claim 18 comprising administering from 10 to 30 mg of clevudine and from 5 to 10 mg of adefovir dipivoxil.

21. The method of claim 18 wherein said infection has an rtL180M mutation.

22. The method of claim 18 wherein said infection has an rtA181T mutation.

23. The method of claim 18 wherein said infection has an rtN236T mutation.

24. A method of treating an adefovir dipivoxil resistant chronic hepatitis B infection having an rtL180M, rtM204I or rtM204V mutation in a patient in need thereof comprising administering to said patient clevudine and adefovir dipivoxil.

25. The method of claim 24, comprising administering from 5 to 100 mg of clevudine and from 5 to 30 mg of adefovir dipivoxil.

26. The method of claim 24 comprising administering from 10 to 30 mg of clevudine and from 5 to 10 mg of adefovir dipivoxil.

27. The method of claim 24 wherein said infection has an rtL180M mutation.

28. The method of claim 24 wherein said infection has an rtA181T mutation.

29. The method of claim 24 wherein said infection has an rtN236T mutation.
